(19) United States
(12) Patent Application Publication
(10) Pub. No.: US 2008/0161901 A1
(43) Pub. Date: Jul. 3, 2008

(54) CATHETER SYSTEM WITH STENT APPARATUS FOR CONNECTING ADJACENT BLOOD VESSELS

(76) Inventors: Richard R. Heuser, Phoenix, AZ (US); James D. Joye, Saratoga, CA (US)

Correspondence Address: KOLISCH HARTWELL, P.C., 520 SW YAMHILL STREET, Suite 200, PORTLAND, OR, 97204, US

(21) Appl. No.: 11/963,460

(22) Filed: Dec. 21, 2007

Related U.S. Application Data
(63) Continuation-in-part of application No. 11/735,629, filed on Apr. 16, 2007.
Application No. 11/735,629 is a continuation-in-part of application No. 11/340,324, filed on Jan. 25, 2006, now Pat. No. 7,374,567.
(60) Provisional application No. 60/887,277, filed on Jan. 30, 2007.

(51) Int. Cl. A61F 2/84; A61F 2/82

(52) U.S. Cl. 623/1.11; 623/1.35

(57) ABSTRACT

A trifurcated stent apparatus for use by a physician includes a main stent for inserting into a trifurcated blood vessel and two side stents. The main stent includes two openings on the side which are the same diameter as the corresponding side stents. The main stent may be configured to receive a first end of a first side stent and a first end of a second side stent, to create a trifurcated stent. Alternatively, the side stents and the main stent may form a single integrated unit. The main stent and side stents can include one-way valves on one or more of the ends. The one or more one-way valves may be opened or closed, depending on whether the physician desires that fluid pass through. While closed, the valve may be configured to allow passage of various cardiovascular instruments, including but not limited to guidewires, catheters, balloons, or any other device used in blood vessel operations, while not allowing the passage of any fluids.

### CROSS REFERENCE TO RELATED APPLICATIONS

This application is a continuation-in-part of application Ser. Nos. 11/340,324, filed Jan. 25, 2006, and 11/735,629, filed Apr. 16, 2007, the disclosures of which are incorporated by reference. This application also claims the benefit of Provisional Application Ser. No. 60/887,277, filed on Jan. 30, 2007, the disclosure of which is incorporated by reference.

### BACKGROUND

This disclosure relates generally to a catheter system for connecting adjacent blood vessels, e.g., an artery and an adjacent vein to adapt the vein for arterial blood flow. More particularly the disclosure concerns a system of two catheters with mating, magnetic tips for creating openings in the artery wall and vein wall to form a fistula connecting the blood vessels. Further, the disclosure relates to a stent apparatus used to bypass a flush occlusion occurring in one passage of a bifurcated vessel or alternatively a trifurcated vessel.

A catheter apparatus and method for arterializing a section of a vein to bypass a clogged artery are shown in U.S. Pat. No. 6,464,665, which is hereby incorporated by reference. The method is used to bypass a stenosis in the artery that obstructs blood flow in a portion of the artery. If the obstructed portion of the artery can be bypassed, blood flow will be restored downstream from the stenosis. A vein running alongside the artery in the obstructed portion of the artery can be used for the bypass.

The catheter apparatus includes one catheter for inserting into the artery and another catheter for inserting into the adjacent vein. The physician maneuvers the tips of both catheters to coincident positions within each blood vessel adjacent one end of the obstructed portion of the artery. The physician then creates an opening from the inside of one blood vessel through the vessel wall and then through the wall of the other blood vessel.

An issue arises in co-locating the openings in the two blood vessels and holding the vessel walls in place to ensure that a channel will be created between the vessels so that blood will flow from one vessel to the other. Another issue arises when connecting adjacent bifurcated vessels having a primary passage and a secondary passage. Sometimes an occlusion occurring in the secondary passage is flush at the origin of the secondary passage, leaving no trace of where the secondary passage begins. In such instances there is no starting point for intervention. An example of where this occurs is at the bifurcation of the femoral artery. In such cases, an occlusion may occur in a side branch off of the profunda. The occlusion must be bypassed, but without obstructing blood flow into the vital profunda femoris. Currently, these situations are only treatable using conventional open surgery.

### SUMMARY OF THE DISCLOSURE

The disclosed system and method provides for creating paired, co-located openings and a consequent fistula between an artery and an adjacent vein to bypass an arterial blockage. The system includes a piercing tool on a first catheter that mates with a receptor on a second catheter to create the co-located openings at one side of the blockage. Magnets incorporated in either or both catheters may be used to draw the piercing tool into the receptor. The piercing tool and receptor typically are provided with complementary, mating contours to draw the piercing tool sufficiently into the receptor to ensure completion of the openings. The openings may be expanded by balloon angioplasty and a stent is typically then installed to interconnect the openings to ensure a fistula is established between the vessels. The process may be repeated at the other side of the arterial blockage to complete the bypass.

Another aspect of the disclosure provides for a bifurcated stent apparatus for use by a physician that includes a main stent for inserting into a bifurcated blood vessel and a side stent. The main stent has an opening on the side which is the same diameter as the side stent. The main stent may be configured to receive a first end of the side stent, to create a bifurcated stent. Alternatively, the side stent and the main stent may form a single integrated unit. The side stent includes a one-way valve on the second end. The one-way valve may be opened or closed, depending on whether the physician desires that fluid pass through. While closed, the valve may be configured to allow passage of various cardiovascular instruments, including but not limited to guidewires, catheters, balloons, or any other device used in blood vessel operations, while not allowing the passage of any fluids.

Yet another aspect of the disclosure provides for a trifurcated stent apparatus for use by a physician that includes a main stent for inserting into a trifurcated blood vessel and two side stents. The main stent includes two openings on the side which are the same diameter as the corresponding side stents. The main stent may be configured to receive a first end of a first side stent and a first end of a second side stent, to create a trifurcated stent. Alternatively, the side stents and the main stent may form a single integrated unit. The main stent and side stents can include one-way valves on the ends. The one-way valves may be opened or closed, depending on whether the physician desires that fluid pass through. While closed, the valve may be configured to allow passage of various cardiovascular instruments, including but not limited to guidewires, catheters, balloons, or any other device used in blood vessel operations, while not allowing the passage of any fluids.

### BRIEF DESCRIPTION OF THE DRAWINGS

FIG. 1 is a partial cross-sectional view showing an obstructed artery, including the obstruction and the area adjacent both ends of the obstruction, and a vein alongside the artery.

FIG. 2 is a cross-sectional view of an embodiment of the present invention in the blood vessels of FIG. 1 with a first catheter with a distal end inserted into the artery and a second catheter with a distal end inserted into the vein, the catheters carrying at their distal ends mating tips, i.e., a piercing tool on the first catheter and a receptor on the second catheter.

FIG. 3 is a cross-sectional view of the vein, artery, and two catheters, as in FIG. 2 with the tips of the catheters mated to create a pair of co-located openings in the walls of the vein and artery for connection of a fistula between the artery and the vein.

FIG. 4 is a cross-sectional view of the vein and artery with a balloon inserted through both openings.

FIG. 5 is a cross-sectional view of the vein and artery with a stent installed through the openings between the vein and artery to maintain a fistula therebetween.

FIG. 6 is a cross-sectional view of a first catheter inserted in the artery and a second catheter inserted in the vein at the other end of the obstruction depicted in FIGS. 1-4, the catheters including mating tips shown in a joined position to create a second pair of co-located openings through the vein and artery walls.

FIG. 7 is a cross-sectional view of the vein and artery with a balloon inserted through the second pair of openings between the vein and the artery.

FIG. 8 is a cross-sectional view of the vein and artery with a second stent installed through the second pair of openings between the vein and artery to maintain a fistula therebetween.

FIG. 9 is a close-up perspective view of the mating tips of the first and second catheters, showing the receptor, which includes a proximal end, a distal opening, and a channel providing a guide surface, and the piercing tool, which includes a needle and a plug encompassing the catheter adjacent the base of the needle, and showing the contours of the plug, needle, and receptor channel that provide for mating between the tips.

FIG. 10 is a piercing tool for use in a second embodiment of the present invention that includes a base and a needle that is offset from the base by an angle.

FIG. 11 illustrates the use of the piercing tool of FIG. 10 in conjunction with a double-balloon catheter to create openings in a vein and an artery.

FIG. 12 illustrates the use of the piercing tool of FIGS. 2, 3, 6, and 9 in conjunction with a double-balloon catheter to create openings in a vein and an artery.

FIG. 13 depicts a main stent having a side opening according to another aspect of the present disclosure.

FIG. 14 depicts a side stent having a one-way valve affixed on one end according to the present disclosure.

FIG. 15 depicts the main stent of FIG. 13 and the side stent of FIG. 14 coupled to one another.

FIG. 16 depicts an alternative embodiment of the present disclosure, where the side stent and main stent form one integrated unit.

FIGS. 17A-E depict the steps of installing a stent apparatus of the present disclosure into a pair of bifurcated vessels.

FIG. 18 depicts a main stent having two side openings according to another aspect of the present disclosure.

FIG. 19 depicts two side stents according to the present disclosure.

FIG. 20 depicts the main stent of FIG. 18 and the two side stents of FIG. 19 coupled to one another.

FIG. 21 depicts an alternative embodiment of the present disclosure, where the two side stents and main stent form one integrated unit.

FIG. 22 depicts a trifurcated stent apparatus of the present disclosure installed into a trifurcated vessel and a neighboring vessel.

FIG. 23 depicts a trifurcated stent apparatus of the present disclosure installed into a trifurcated vessel and a neighboring trifurcated vessel below a knee joint in a lower leg.

### DETAILED DESCRIPTION OF THE PREFERRED EMBODIMENTS

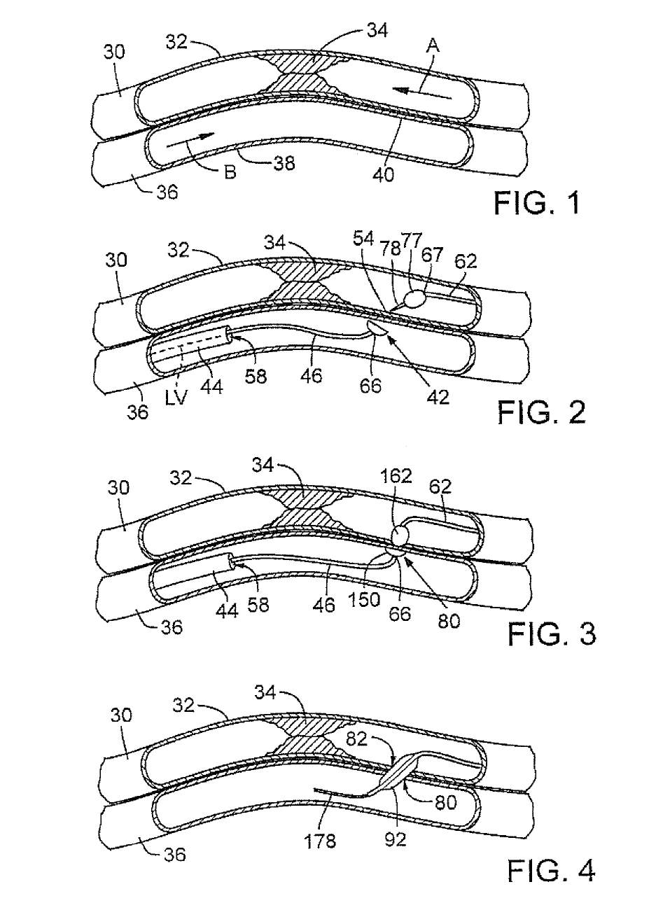

As shown in FIG. 1, an artery 30, formed by an artery wall 32, has a blood flow, indicated by arrow A, that is partially or totally blocked by an obstruction or occlusion 34, typically formed by plaque. A vein 36 roughly similar in dimension to artery 30 lies alongside and generally parallel to artery 30. Vein 36, formed by a vein wall 38, includes, in the area proximal to occlusion 34, a portion 40 in close proximity to artery 30 that the physician has selected as a venous site for creating a fistula between artery 30 and vein 36. The normal blood flow through vein 36 would be in the direction indicated by arrow B.

An embodiment of the invented system, indicated generally at 42 in FIG. 2, is a catheter apparatus that includes a first catheter 62 and a second catheter 44. In FIG. 2, the first catheter is in the artery and the second catheter is in the vein, but this can be reversed. Similarly, the first catheter in the artery is shown upstream from occlusion 34, but this may alternatively be reversed to begin the procedure downstream from the occlusion and proceeding afterwards to the upstream side.

Second catheter 44 may include at least one lumen 58 which runs generally parallel to a longitudinal axis LV of catheter 44. A wire 46 may be inserted through lumen 58. Typically, wire 46 has an outer diameter of 0.035-inches, but any suitable dimension may be used. Wire 46 may be controllable by the physician in position relative to catheter 44. Wire 46 may be a guidewire for catheter 44, or a separate guidewire may be used, with other lumens in catheter 44 providing the channel for the separate guidewire.

As shown in FIG. 2, first catheter 62 of catheter apparatus 42 includes a distal end 67 that the physician may insert into artery 30 for positioning adjacent arterial fistula site 54. First catheter 62 may include one or more lumens running generally parallel to a longitudinal axis of catheter 62. First catheter 62 may be guided along a guidewire or may itself be a guidewire, typically with an outer diameter of 0.035-inches, although any suitable dimension may be used. First catheter 62 preferably is hollow.

A piercing tool 77 that includes a sharp needle 78, may be selectively deployed, as shown in FIGS. 2 and 3, or withdrawn into the lumen of catheter 62. Needle 78 is preferably withdrawn while catheter 44 is maneuvered to the fistula site so as not to cause trauma to the blood vessel wall.

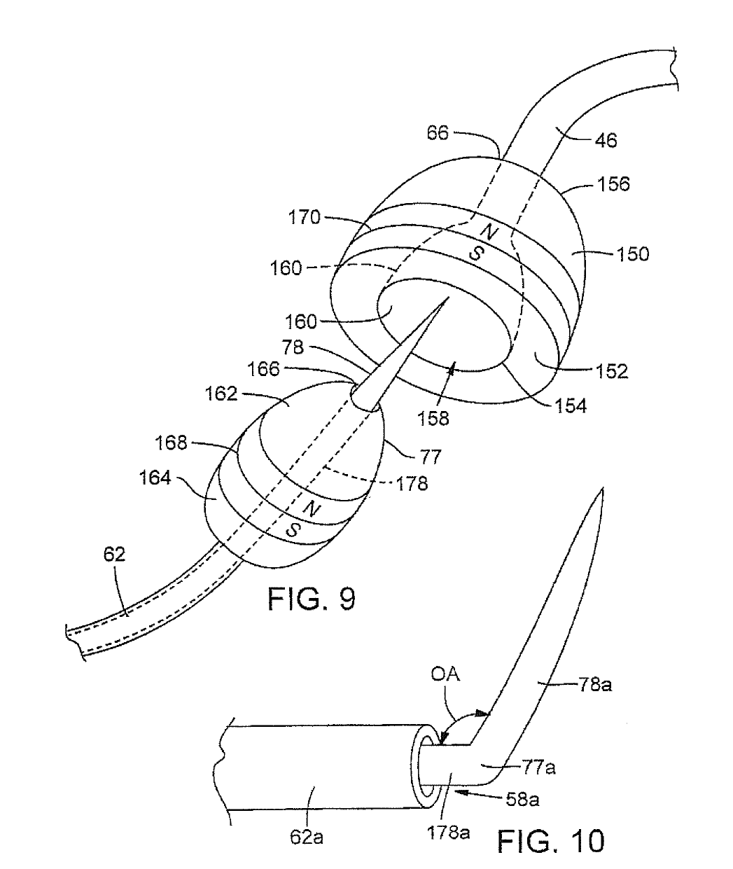

As best seen in FIG. 9, needle 78 may be disposed at the distal end of a wire 178 disposed in the lumen of catheter 62. The physician can control the positioning of wire 178 and needle 78 relative to catheter 62. Guidewire 46 may include a receptor 150, such as substantially cup-shaped socket 152. Receptor 150 includes a distal opening 154, preferably circular, and a proximal end 156. Receptor 150 includes a channel 158 leading from opening 154 toward proximal end 156. Channel 158 preferably narrows in a direction from opening 154 toward proximal end 156. Channel 158 is defined by an inner surface 160 that provides a guide surface for needle 78 that directs the needle toward proximal end 156 of receptor 150. Channel 158 may be substantially conical, or have such other shape as tends to mate with, and guide piercing tool 77 into receptor 150.

Piercing tool 77 on catheter 62 preferably includes a plug 162 provided with an outer contour that narrows from a proximal end 164 toward a distal end 166. Plug 162 preferably mates with channel 158 in receptor 150. Plug 162 preferably encompasses catheter 62 adjacent the distal end of the catheter. As seen in FIGS. 2, 3, and 9, the piercing tool and the receptor have a complementary configuration that supports their mating together.

Typically, piercing tool 77 will include a magnet with one pole oriented toward the distal end of the tool, while receptor 150 will include a magnet with the opposite pole oriented toward the distal end of the receptor which will draw the needle into the receptor. For example, the magnets may be annular rings or donuts and formed of a strong permanent magnet material suitable for the intended use.

A typical arrangement, shown in FIG. 9, is that plug 162 includes a first magnet 168 generally in a donut shape and having a north pole N positioned distally with respect to a south pole S. Typically magnet 168 is spaced from the distal end of plug 162. A second magnet 170 may be disposed on, or form an integral part of receptor 152, preferably adjacent distal opening 154 of socket 152. Second magnet 170 may be arranged with a south pole S distal of a north pole N to attract magnet 168 when the tips of the two catheters are in proximity, e.g., with each catheter in an adjacent blood vessel. Alternatively or in addition one or more magnets may be arranged in various locations on plug 162 and/or needle 78 and on or in receptor 150, e.g., adjacent proximal end 156, with the poles arranged to draw piercing tool 77 into receptor 150.

As shown in FIGS. 3 and 4, after creating openings 80, 82 with a tool such as needle 78, the physician withdraws catheter 62 from the fistula site, leaving wire 178 in place, and a balloon 92 may be inserted over wire 178 and through openings 80, 82 and inflated to enlarge the openings. Balloon 92 may include radiopaque markers and may be inflated with a solution containing a radiopaque dye or contrast to allow the physician to radiographically monitor and adjust the position of the balloon before, during, and after inflation.

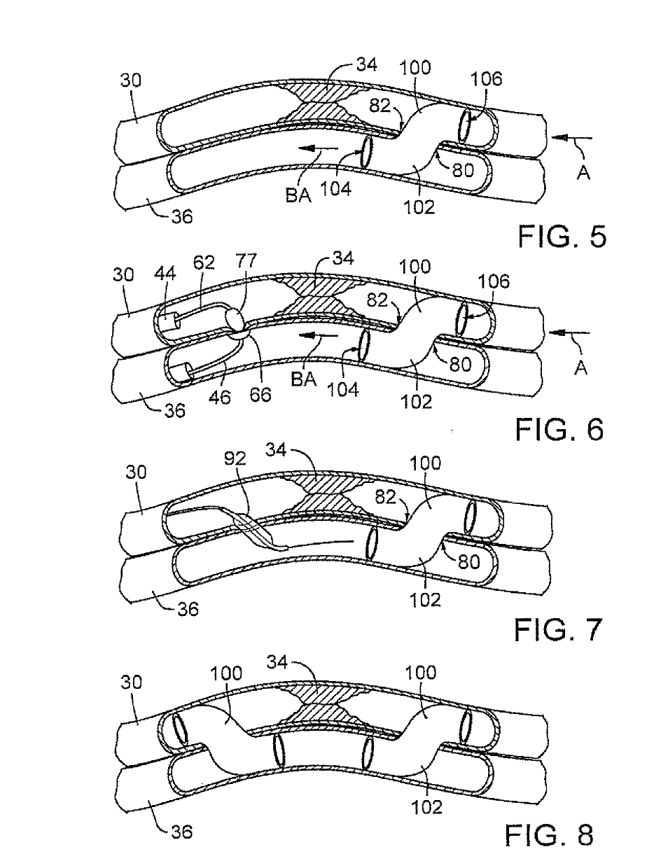

As shown in FIG. 5, a device for maintaining an open, leak-free connection between openings 80 and 82, such as stent 100, is inserted through the openings. Stent 100 includes a frame 102 having two open ends 104 and 106 that preferably create leak-free couplings to the inside of artery 30 and vein 36. With openings 80, 82 connected to form a fistula, vein 36 is arterialized, and blood flows from artery 30 into vein 36 in the direction indicated by arrows A and BA.

Stent 100 is typically a short, covered stent, such as the Hemobahn stent made by WL Gore & Associates.

As shown in FIGS. 6, 7, and 8 a second pair of co-located openings may be created, and a stented fistula established therebetween, using essentially the same catheter system and method as described for FIGS. 1-5 and 9. FIG. 6 illustrates that the first catheter with the piercing tool preferably is inserted into the artery and the openings created from the artery into the vein. Alternatively the openings may be created from the vein into the artery.

An alternative embodiment for the piercing tool in shown in FIG. 10. This tool 77a may be used with a metal guidewire 62a that preferably includes a lumen 58a. An inner wire 178a may be inserted in lumen 58a, providing a base for a needle 78a. The coupling between the needle and base incorporates a curvature such that the needle is nominally offset from the base by an angle OA, typically between about 30-degrees and about 90-degrees. Inner wire 178a is typically made of a sufficiently rigid material, such as nitinol and/or stainless steel, as to maintain the offset angle as the needle is used to pierce blood vessels. Guidewire 62a is preferably formed of a sufficiently rigid material such that when needle 78a is retracted into lumen 58a, the curvature between the needle and the base is overcome and the needle temporarily aligns with the base in a non-traumatic configuration. Inner wire 178a may have an outer diameter of 0.010, 0.014, 0.018, or 0.021-inches, or such other dimension as is suited to the particular application.

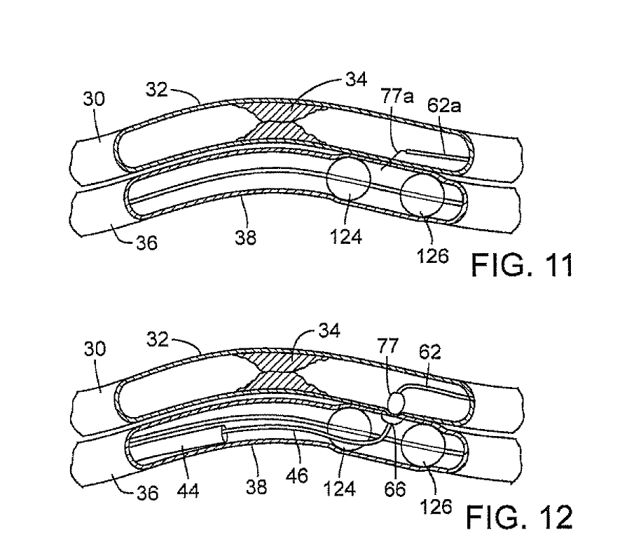

As shown in FIG. 11, piercing tool 77a may be inserted in artery 30, typically while withdrawn into the catheter 62a while maneuvering to the fistula site. Piercing tool 77a may be used in conjunction with a catheter having two balloons 124 and 126 that are inserted in vein 36. In such case, the catheter tips are maneuvered to opposing sides of the proposed fistula site and balloons 124 and 126 are inflated to press the vein wall against the artery wall. Also, fluid may be injected into the sealed-off area to further press the two blood vessel walls together. Then piercing tool 77a is deployed and maneuvered through the artery and then the vein wall to create openings for forming the fistula as for the embodiments described above.

FIG. 11 depicts the piercing tool and the balloon catheter in different vessels. Alternatively, piercing tool 77a may be inserted in the same blood vessel with the balloon catheter. In such an embodiment, the balloons are preferably independently inflatable, and typically the distal balloon 124 is inflated first to stop blood flow. Then, piercing tool 77a is maneuvered to the fistula site in a manner similar to that for the previously described embodiment, typically with the piercing tool withdrawn into the guidewire to the non-traumatic configuration.

With the piercing tool at the fistula site, the proximal balloon 126 is inflated to seal off the fistula site and also to press the vein against the artery. Then, piercing tool 77a is deployed at the end of guidewire 62a and maneuvered by the physician to create the openings from one blood vessel, through both walls, to the other blood vessel.

In either case, piercing tool 77a may be used to create multiple pairs of co-located openings which are then stented to arterialize a portion of the vein to bypass a blockage using a similar method as described above for the embodiment of FIGS. 1-9.

As shown in FIG. 12, the double balloon catheter may also be used in conjunction with the catheters 44 and 62 that include the mating tips. In this embodiment, the double balloon catheter helps to control blood flow at the planned fistula site and to press the blood vessel walls together to assist in the mating of the tips. The fistula creation otherwise proceeds in a similar manner as for the embodiment of FIGS. 1-9.

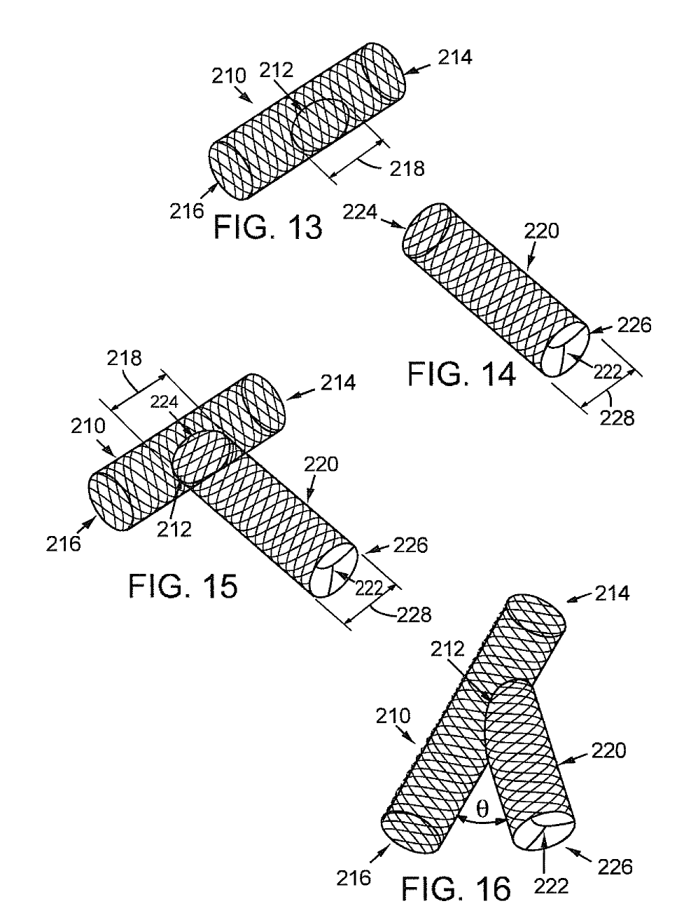

Referring now to FIG. 13, a main stent 210 is shown having a side opening 212, a proximal end 214, a distal end 216, and a side opening diameter 218. The main stent 210 may be manipulable between a nominal diameter and an active diameter. The nominal diameter is smaller than the diameter of a blood vessel through which the main stent 210 traverses. The active diameter is substantially equal to the diameter of the blood vessel.

The main stent 210 may be constructed out of any suitable material. In one embodiment, the main stent 210 is metallic. In another embodiment, the main stent 210 is comprised at least in part of self-expanding nitinol. The main stent 210 may be a porous stent used for placeholding. Additionally and alternatively, the main stent 210 may be covered in an impermeable membrane (e.g., polytetrafluoroethylene).

FIG. 14 depicts a side stent 220 having a proximal end 224, a distal end 226, and a one-way valve 222 adjacent to the distal end 226. The side stent 220 may be manipulable between a second nominal diameter and a second active diameter 228. The second nominal diameter is smaller than the diameter of a blood vessel through which the side stent 220 traverses. The second active diameter 228 is substantially equal to the diameter of the blood vessel. The second active diameter 228 may additionally be substantially equivalent to the side opening diameter 218 of the main stent 210.

The side stent 220 may be constructed out of any suitable material. In one embodiment, the side stent 220 is metallic. In another embodiment, the side stent 220 is comprised at least in part of self-expanding nitinol. The side stent 220 may be a porous stent used for placeholding. Additionally and alternatively, the side stent 220 may be covered in an impermeable membrane (e.g., polytetrafluoroethylene).

FIG. 15 depicts the main stent 210 and the side stent 220 coupled together. The side stent 220 is shown with its proximal end 224 coupled to the opening 212 of the main stent 210. Coupling the main stent 210 and the side stent 220 in such a manner effectively creates a bifurcated stent apparatus.

While FIGS. 13-15 depict the main stent 210 and the side stent 220 as separated and arranged generally perpendicular to one another, other embodiments are possible. In one example depicted in FIG. 16, the main stent 210 and the side stent 220 comprise a single integrated unit. In such an embodiment, the side stent 220 may retract towards the main stent 210 while the integrated unit travels through blood vessels, only to be extended once the main stent 210 is in place and the fistula to an adjacent blood vessel is created.

In another example, the side stent 220 is configured to extend away from the main stent 210 at an angle θ not perpendicular to the main stent, as seen in FIG. 16. It should be understood that the side stent 220 may extend away from the main stent at any angle θ between 0° (which would require a bend in the side stent 220) and 180°.

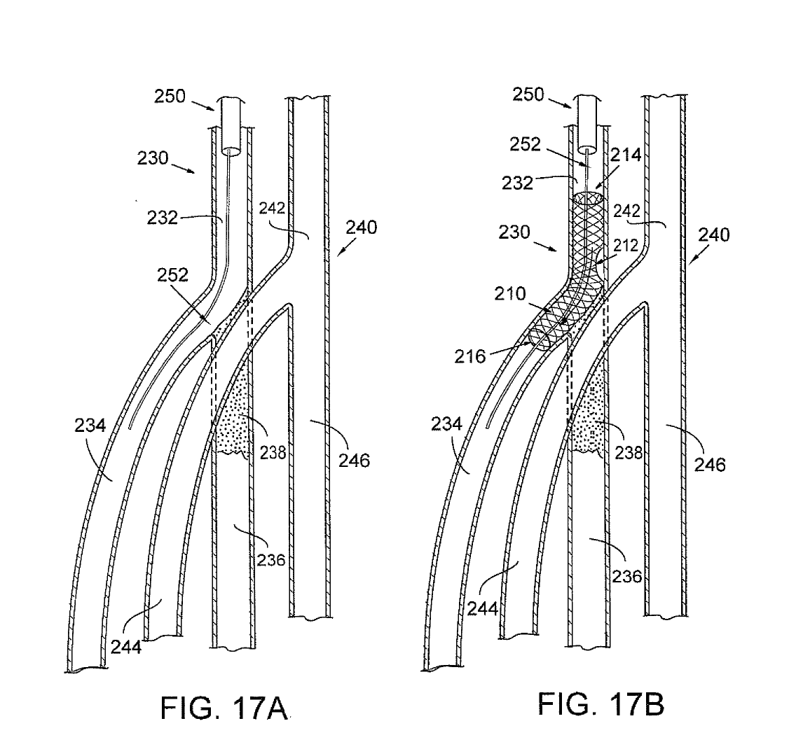

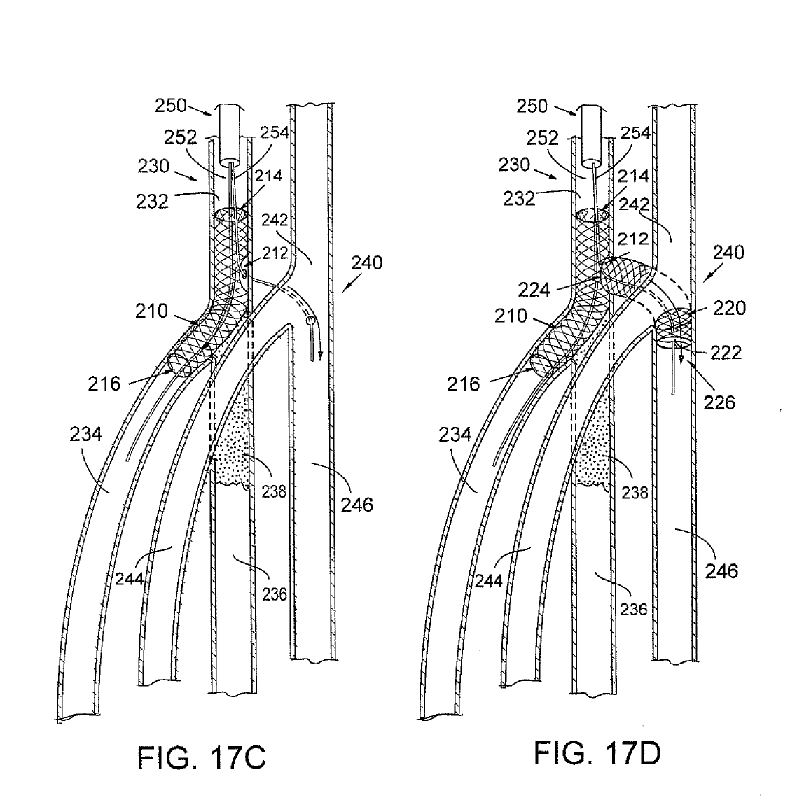

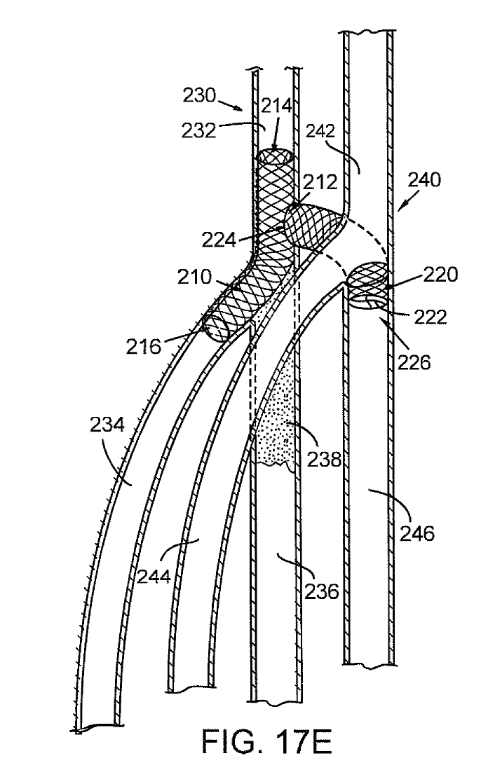

FIGS. 17A-E depict one possible vessel arrangement where a stent apparatus of the present disclosure may be used. The arrangement includes a first bifurcated blood vessel 230 and a second bifurcated blood vessel 240. The first bifurcated blood vessel 230 comprises a first common portion 232, a first primary passage 234, and a first secondary passage 236. The second bifurcated blood vessel 240 comprises a second common portion 242, a second primary passage 244, and a second secondary passage 246.

In this particular scenario, occlusion 238 has entirely blocked blood flow through the first secondary passage 236. Additionally, occlusion 238 is flush with the origin of first secondary passage 236. In such instances, a physician may experience difficulties in accessing the first secondary passage 236.

Without being able to access the first secondary passage 236, the physician cannot create a fistula from the first secondary passage 236 to an adjacent blood vessel for percutaneous bypass, as described in U.S. Pat. No. 6,464,665 or in the systems discussed above.

In some cases, the first bifurcated blood vessel 230 may be the femoral artery. In such cases the first primary passage 234 is the profunda and the first secondary passage 236, seen blocked with occlusion 238, may be any number of branched passages. Similarly, the second bifurcated blood vessel 240 may be the femoral vein, with the second primary passage 244 being the deep femoral vein. Of course, the present disclosure is not limited to treating the aforementioned vessels; any two adjacent bifurcated blood vessels may be treated with the disclosed stent apparatus.

In FIG. 17A, a catheter 250 is seen in the first common portion 232 of the first bifurcated vessel 230. A first guidewire 252 extends from the catheter down the first primary passage 234.

In FIG. 17B, a main stent 210 has been traversed down the first guidewire 252 and is seen in its active diameter positioned so that the distal end 216 of the main stent 210 extends into the first primary passage 234. The side opening 212 of the main stent 210 is positioned adjacent to a site intended for a fistula between the first bifurcated vessel 230 and the second bifurcated vessel 240.

Referring now to FIG. 17C, a second guidewire 254 is seen extending from the catheter 250, through the wall of the first bifurcated blood vessel 230 in the area surrounded by opening 212, through the wall of the second bifurcated blood vessel 240, and into the lumen of the second secondary passage 246.

In FIG. 17D, a side stent 220 has been advanced down the second guidewire 254. The side stent in its active diameter extends from the opening 212 of the main stent, through the wall of the first bifurcated blood vessel 230, through the wall of the second bifurcated blood vessel 240, and into the second secondary passage 246.

It should be understood that while the distal end 226 of side stent 220 is seen extending into the second secondary passage 246, the side stent 220 may alternatively extend into the second primary passage 244, or even into the second common passage 242. The proximal end 224 of the side stent in its active diameter may be coupled to the opening 212 of the main stent 210.

Alternatively, in an embodiment where the main stent 210 and the side stent 220 form a single integrated unit, the side stent 220 may already be coupled to the main stent. In such cases, the side stent 220 may be retracted towards the main stent 210 during traversal through blood vessels. Once the main stent 210 is in place, the side stent 220 may be telescoped or otherwise extended away from the main stent 210 and into the second bifurcated blood vessel 240.

The side stent 220 of FIG. 17D has a one-way valve 222 disposed adjacent to the distal end 226. This valve may be manipulable between an open position, which would allow fluid (e.g., blood) to pass into the second secondary passage 246, and a closed position, which prevent fluid from passing into the second secondary passage 246.

In one embodiment, a guidewire may be extended through the one-way valve 222, even when the valve 222 is in the closed position, without allowing any extraneous fluid to pass into the second secondary passage 246, as seen in FIG. 17D. The one-way valve 222 could additionally or alternatively be configured to allow the passage of numerous instruments while in the closed position, without allowing the passage of any fluid. These instruments could include but are not limited to catheters, catheters with stents, stents, balloons, or any other instrument used in percutaneous procedures.

In such an embodiment, a physician may traverse additional stents or endografts through the one-way valve 222 and position them further down the second secondary passage 246. Once these additional devices have been placed, the physician could then open the one-way valve to allow blood flow into the second secondary passage 246.

FIG. 17E depicts the bifurcated stent apparatus in its final position between the first bifurcated blood vessel 230 and the second bifurcated blood vessel 240. The first guidewire 252 and the second guidewire 254 have been removed.

A fistula may be created in the second secondary passage 246 downstream from the side stent 220, the fistula going from the second secondary passage back into the first secondary passage 236 at a point downstream from the occlusion 238. In such an arrangement, the blood flowing through the opening 212 is directed into the second secondary passage 246, bypassing the occlusion 238, and then is directed back into the first secondary passage 236.

At no point during the procedure depicted in FIGS. 17A-E has blood flow down the first primary passage 234 been obstructed. This is vital when the first primary passage 234 is the profunda femoris.

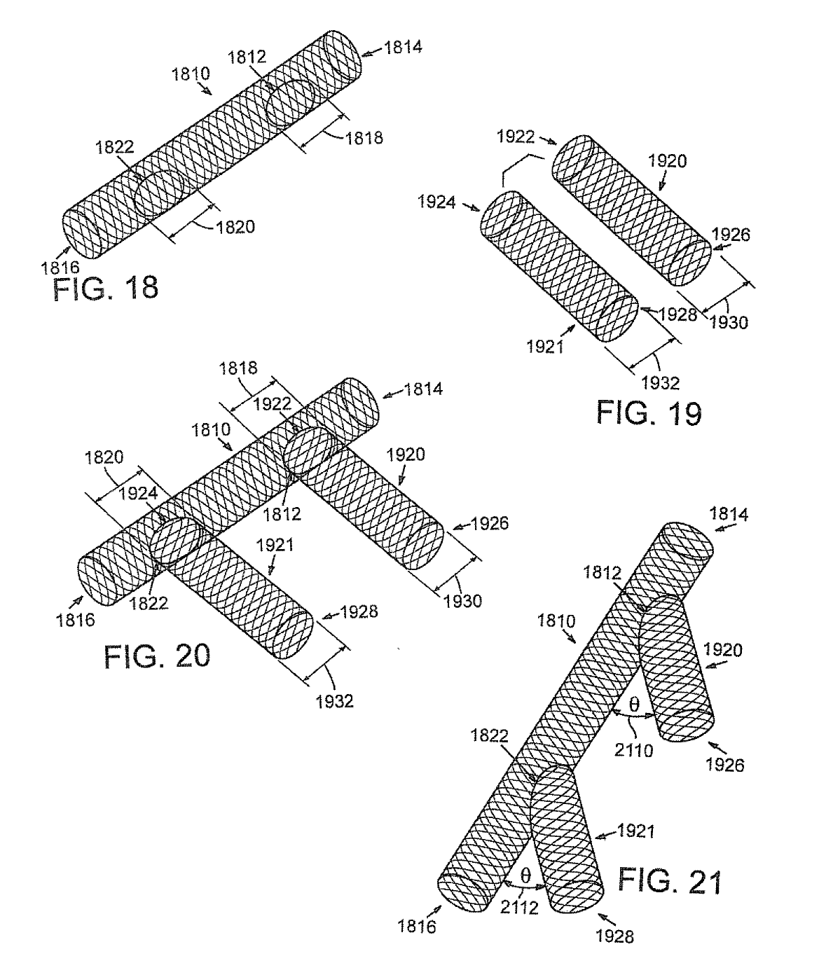

Referring now to FIG. 18, a main stent 1810 is shown having a first side opening 1812, a second side opening 1822, a proximal end 1814, a distal end 1816, a first side opening diameter 1818, and a second side opening diameter 1820. The main stent 1810 may be manipulable between a nominal diameter and an active diameter. The nominal diameter is smaller than the diameter of a blood vessel through which the main stent 1810 traverses. The active diameter is substantially equal to the diameter of the blood vessel.

The main stent 1810 may be constructed out of any suitable material. In one embodiment, the main stent 1810 is metallic. In another embodiment, the main stent 1810 is comprised at least in part of self-expanding nitinol. The main stent 1810 may be a porous stent used for placeholding. Additionally and alternatively, the main stent 1810 may be covered in an impermeable membrane (e.g., polytetrafluoroethylene).

FIG. 19 depicts a first side stent 1920 having a proximal end 1922 and a distal end 1926. The first side stent 1920 may be manipulable between a second nominal diameter and a second active diameter 1930. The second nominal diameter is smaller than the diameter of a blood vessel through which the side stent 1920 traverses. The second active diameter 1930 is substantially equal to the diameter of the blood vessel. The second active diameter 1930 may additionally be substantially equivalent to the side opening diameter 1818 of the main stent 1810.

FIG. 19 also depicts a second side stent 1921 having a proximal end 1924 and a distal end 1928. The second side stent 1921 may be manipulable between a third nominal diameter and a third active diameter 1932. The third nominal diameter is smaller than the diameter of a blood vessel through which the side stent 1921 traverses. The third active diameter 1932 is substantially equal to the diameter of the blood vessel. The third active diameter 1932 may additionally be substantially equivalent to the side opening diameter 1820 of the main stent 1810. Therefore, the second side stent 1920 and the third side stent 1921, can be similar in diameter and/size or different depending on what vessels they are designed to be installed within.

Side stents 1920 and 1921 may be constructed out of any suitable material. In one embodiment, the side stents 1920 and 1921 are metallic. In another embodiment, the side stents 1920 and 1921 are comprised at least in part of self-expanding nitinol. The side stents 1920 and 1921 may be porous stents used for placeholding. Additionally and alternatively, the side stents 1920 and 1921 may be covered in an impermeable membrane (e.g., polytetrafluoroethylene).

FIG. 20 depicts the main stent 1810 and the side stents 1920 and 1921 coupled together. The first side stent 1920 is shown with its proximal end 1922 coupled to the opening 1812 of the main stent 1810. The second side stent 1921 is shown with its proximal end 1924 coupled to the opening 1820 of the main stent 1810. Coupling the main stent 1810, the first side stent 1920 and the second side stent 1921 in such a manner effectively creates a trifurcated stent apparatus.

While FIGS. 18-20 depict the main stent 1810 and the first and second side stents 1920 and 1921 as separated and arranged generally perpendicular to one another, other embodiments are possible. In one example depicted in FIG. 21, the main stent 1810 and the first and second side stents 1920 and 1921 comprise a single integrated unit. In such an embodiment, the first and second side stents 1920 and 1921 may retract towards the main stent 1810 while the integrated unit travels through blood vessels, only to be extended once the main stent 1810 is in place and the fistula to an adjacent blood vessel is created, thereby having the side stents 1920 and 1921 extend into side vessels of a trifurcated vessel while the main stent 1810 extends through the main vessel and an adjacent blood vessel.

In another example, the first side stent 1920 is configured to extend away from the main stent 1810 at a first angle θ (2110) not perpendicular to the main stent, as seen in FIG. 21. Similarly, the second side stent 1921 is configured to extend away from the main stent 1810 at a second angle θ (2112) not perpendicular to the main stent, as seen in FIG. 21. It should be understood that side stents 1920 and 1921 may extend away from the main stent 1810 at any first and second angle θ between 0° (which would require a bend in the side stents 1920 and 1921) and 180°.

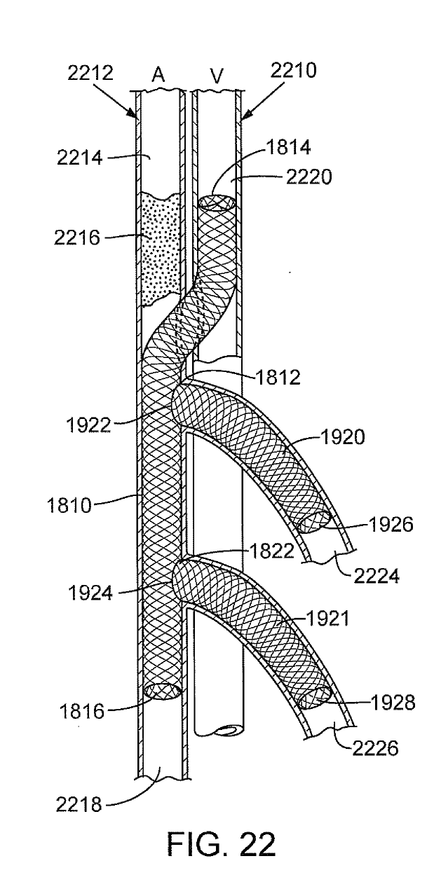

FIG. 22 depicts one possible vessel arrangement where a stent apparatus of the present disclosure may be used. The arrangement includes a trifurcated blood vessel 2212 (e.g., an artery) and second blood vessel (e.g., a vein) 2210. The trifurcated blood vessel 2212 comprises a common portion 2214, a first primary passage 2218, a secondary passage 2224, and a tertiary passage 2226. The second blood vessel 2210 comprises a second primary passage 2220. The second blood vessel can be similarly bifurcated (as was shown in FIGS. 17A-E in the bifurcated example) or even trifurcated. In this particular scenario, occlusion 2216 has entirely blocked blood flow through the common portion 2214 of trifurcated blood vessel 2212. Stent 1810 has been placed with proximal end 1814 in the second primary passage 2220 of the second blood vessel 2210. Stent 1810 extends through the wall of the second blood vessel 2210 and through the wall of the first blood vessel 2212 forming a fistula between the vessels. Distal end 1816 of stent 1810 extends through the common portion 2214 past the junctures with the secondary and tertiary passages 2224 and 2226. First side stent 1920 extends through secondary passage 2224 and second side stent 1921 extends through tertiary passage 2226. In such a manner, the placed stent makes for a more structurally sound valve/vessel region. Furthermore, if all three branches of a trifurcated vessel are diseased, by connecting the bypass arterialized vein to three vessels instead of one vessel, patency is improved (e.g., if one branch occludes, the flow can likely continue through one of the other branches/vessels).

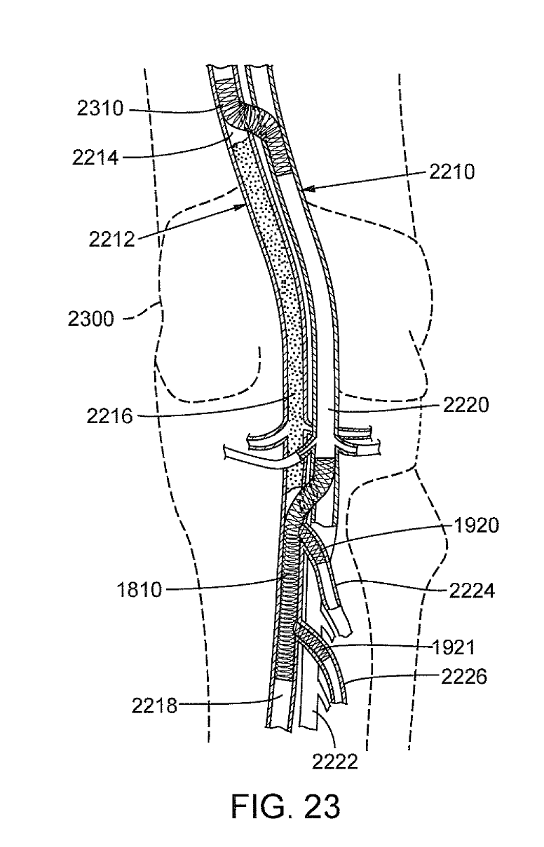

In some cases (as illustrated in FIG. 23), the first trifurcated blood vessel 2212 may be the popliteal artery, the second vessel 2210 may be the popliteal vein, and an occlusion 2216 may exist in common portion 2214 of the first trifurcated blood vessel 2212 in the knee region 2300. In such cases, a stent 2310 can be placed above the occlusion 2216 to form a fistula between the popliteal artery and the popliteal vein. Below occlusion 2216, trifurcated stent 1810 can be placed forming a second fistula between the popliteal artery and the popliteal vein, thereby arterializing the popliteal vein in the region around occlusion 2216 in the popliteal artery 2212. First side stent 1920 can extend into the anterior tibial artery 2224, second side stent 1921 can extend into the peroneal/fibular artery 2226, with the distal end 1816 of stent 1810 extending into the posterior tibial artery. Venous blood flow that previously passed through the now arterialized portion of the popliteal vein is now blocked by stent 1810. Instead of flowing through the primary passage 2222 of the popliteal vein (or via the posterior or anterior tibial veins) into the common passage 2220 of the popliteal vein, the venous flow in the lower leg will follow an alternative path to get to the common passage 2220 of the popliteal vein (e.g. via the small saphenous vein which connects with common passage 2220 at or above the knee joint), therefore connecting venous flow again above the arterialized portion of the popliteal vein.

Of course, the present disclosure is not limited to treating the aforementioned vessels; any two adjacent bifurcated, trifurcated, or any combination thereof blood vessels may be treated with the disclosed stent apparatuses.

As previously described and illustrated in FIGS. 17A-E with the bifurcated stent, guidewires can be utilized to place the trifurcated stent in position in a similar manner incorporating technologies disclosed herein and incorporated by reference herein.

One-way valves can be incorporated into any of the ends of the trifurcated stent disclosed. Such valves may be manipulable between an open position, which would allow fluid (e.g., blood) to pass, and a closed position, which prevents fluid from passing through. In one embodiment, a guidewire may be extended through one or more one-way valves of the trifurcated stent, even when the one or more valves are in the closed position, without allowing any extraneous fluid to pass through. The one or more one-way valves could additionally or alternatively be configured to allow the passage of numerous instruments while in the closed position, without allowing the passage of any fluid. These instruments could include but are not limited to catheters, catheters with stents, stents, balloons, or any other instrument used in percutaneous procedures.

In such an embodiment, a physician may traverse additional stents or endografts through the one or more one-way valves and position them further upstream or downstream. Once these additional devices have been placed, the physician could then open the one-way valve to allow blood to flow through the stent.

It is believed that the following claims particularly point out certain combinations and subcombinations that are directed to one of the disclosed disclosures and are novel and non-obvious. Disclosures embodied in other combinations and subcombinations of features, functions, elements and/or properties may be claimed through amendment of the present claims or presentation of new claims in this or a related application. Such amended or new claims, whether they are directed to a different disclosure or directed to the same disclosure, whether different, broader, narrower or equal in scope to the original claims, are also included within the subject matter of the disclosures of the present disclosure.

We claim:

1. A trifurcated stent apparatus for connecting a trifurcated blood vessel having an occlusion in a first primary path, with a second blood vessel having a second primary path, the apparatus comprising:
a main stent including a first opening in a side having a first diameter, a second opening in the side having a second diameter, a first proximal end and a first distal end, the main stent being insertable into a position wherein the first proximal end is extendable into the second primary path of the second blood vessel forming a fistula and the first opening is adjacent to a site within the trifurcated blood vessel having an opening to a first side vessel of the trifurcated blood vessel and the second opening is adjacent to a site within the trifurcated blood vessel having an opening to a second side vessel of the trifurcated blood vessel;
a first side stent having a third diameter substantially equal to the first diameter, a second proximal end and a second distal end, the first side stent being insertable to a position wherein the second proximal end is adjacent to the first opening, and the second distal end extends into the first side vessel of the trifurcated blood vessel; and
a second side stent having a fourth diameter substantially equal to the second diameter, a third proximal end and third distal end, the second side stent being insertable to a position wherein the third proximal end is adjacent to the second opening, and the third distal end extends into the second side vessel of the trifurcated blood vessel.

2. The stent apparatus of claim 1, wherein the first proximal end includes a one-way valve.

3. The stent apparatus of claim 2, wherein the one-way valve is configured to allow a wire to pass without allowing fluid to pass.

4. The stent apparatus of claim 2, wherein the one-way valve is configured to allow a catheter to pass without allowing fluid to pass.

5. The stent apparatus of claim 2, wherein the one-way valve is configured to allow a catheter carrying a constricted stent to pass without allowing any fluid to pass.

6. The stent apparatus of claim 2, wherein the one-way valve is configurable between an open position and a closed position, wherein no fluid may pass while the valve is in the closed position, and fluid may pass when the valve is in the open position.

7. The stent apparatus of claim 1, wherein the first side stent is between 2 cm and 5 cm from the second proximal end to the second distal end.

8. The stent apparatus of claim 1, wherein the second side stent is between 2 cm and 5 cm from the third proximal end to the third distal end.

9. The stent apparatus of claim 1, wherein the first and second openings are radiopaque.

10. The stent apparatus of claim 1, wherein the main stent and the first and second side stents are comprised of self-expanding nitinol.

11. The stent apparatus of claim 1, wherein the first side stent is coupled at the second proximal end to the first opening in the side of the main stent.

12. The stent apparatus of claim 1, wherein the second side stent is coupled at the third proximal end to the second opening in the side of the main stent.

13. The stent apparatus of claim 1, wherein the main stent and the first and second side stents are covered in an impermeable membrane.

14. The stent apparatus of claim 1, wherein the second distal end of the first side stent includes a one-way valve.

15. The stent apparatus of claim 1, wherein the third distal end of the second side stent include a one-way valve.

16. The stent apparatus of claim 1, wherein the first distal end includes a one-way valve.

17. A catheter system for creating and maintaining a fistula between a first trifurcated blood vessel having an occlusion in a first primary path, with a second blood vessel having a second primary path, the system comprising:
a first catheter having a distal end insertable to a position wherein the distal end is adjacent a site within the first blood vessel for the fistula, the first catheter including a piercing tool adjacent the distal end;
a second catheter having a distal end insertable to a position wherein the distal end is adjacent a site within the second blood vessel for the fistula, the second catheter including adjacent the distal end a receptor having a distal opening, a proximal end, and a guide surface disposed between the distal opening and the proximal end;
one or more magnets disposed on at least one of the catheters to draw the piercing tool along the guide surface of the receptor;
a main stent including a first opening in a side having a first diameter, a second opening in the side having a second diameter, a first proximal end and a first distal end, the main stent being insertable into a position wherein the first proximal end is extended into the second primary path of the second blood vessel forming a fistula and the first opening is adjacent to a site within the trifurcated blood vessel having an opening to a first side vessel of the trifurcated blood vessel and the second opening is adjacent to a site within the trifurcated blood vessel having an opening to a second side vessel of the trifurcated blood vessel;
a first side stent having a third diameter substantially equal to the first diameter, a second proximal end and a second distal end, the first side stent being insertable to a position wherein the second proximal end is adjacent to the first opening, and the second distal end extends into the first side vessel of the trifurcated blood vessel; and
a second side stent having a fourth diameter substantially equal to the second diameter, a third proximal end and third distal end, the second side stent being insertable to a position wherein the third proximal end is adjacent to the second opening, and the third distal end extends into the second side vessel of the trifurcated blood vessel.

18. The catheter system of claim 17, wherein the main stent and the first and second side stents are comprised of self-expanding nitinol and covered in an impermeable membrane.

19. The catheter system of claim 17, wherein the first and second openings are radiopaque.

20. A method of creating and maintaining a fistula between a first trifurcated blood vessel having an occlusion in a first primary path, with a second blood vessel having a second primary path, the method comprising:
inserting a first catheter having a distal end to a position wherein the distal end is adjacent the site within the first trifurcated blood vessel for the fistula, the first catheter including a piercing tool adjacent the distal end;
inserting a second catheter having a distal end to a position wherein the distal end is adjacent a site within the second bifurcated blood vessel for the fistula, the second catheter including adjacent the distal end a receptor having a distal opening, a proximal end, and a guide surface disposed between the distal opening and the proximal end;
creating the fistula by drawing the piercing tool along the guide surface of the receptor using one or more magnets disposed on at least one of the catheters;
inserting a main stent into the first trifurcated blood vessel, the main stent having a first opening in a side having a first diameter, a second opening in the side having a second diameter, a first proximal end and a first distal end, whereby upon insertion the first proximal end is extended into the second primary path of the second blood vessel forming a fistula and the first opening is adjacent to a site within the trifurcated blood vessel having an opening to a first side vessel of the trifurcated blood vessel and the second opening is adjacent to a site within the trifurcated blood vessel having an opening to a second side vessel of the trifurcated blood vessel;
placing a first side stent at least partially into the first side vessel, the first side stent having a third diameter substantially equal to the first diameter, a second proximal end and a second distal end; and
placing a second side stent at least partially into the second side vessel, the second side stent having a fourth diameter substantially equal to the second diameter, a third proximal end and third distal end.

21. The method of claim 20, wherein placing a first side stent comprises inserting the first side stent to a position wherein the second proximal end is adjacent to the first opening, and the second distal end extends into the first side vessel of the trifurcated blood vessel;

22. The method of claim 20, wherein placing a second side stent comprises inserting the second side stent to a position wherein the third proximal end is adjacent to the second opening, and the third distal end extends into the second side vessel of the trifurcated blood vessel.

23. The method of claim 20, wherein the first side stent is retractable and coupled with the first opening, the second side stent is retractable and coupled with the second opening, and upon inserting the main stent into the first trifurcated blood vessel the first and second side stents are retracted towards the main stent.

24. The method of claim 23, wherein placing the first side stent comprises extending the first side stent, whereby upon extension the second distal end extends into the first side vessel.

25. The method of claim 23, wherein placing the second side stent comprises extending the second side stent, whereby upon extension the third distal end extends into the second side vessel.
